(19) United States
(12) Patent Application Publication
(10) Pub. No.: US 2007/0264287 A1
(43) Pub. Date: Nov. 15, 2007

(54) Methods for Increasing the Immune Response in an Animal

(76) Inventors: Steven C. Zicker, Lawrence, KS (US); Inke Paetau-Robinson, Auburn, KS (US)

Correspondence Address: COLGATE-PALMOLIVE COMPANY, 909 RIVER ROAD, PISCATAWAY, NJ, 08855, US

(21) Appl. No.: 11/753,373

(22) Filed: May 24, 2007

Related U.S. Application Data
(63) Continuation of application No. PCT/US05/42805, filed on Nov. 23, 2005.
(60) Provisional application No. 60/630,972, filed on Nov. 24, 2004.

(51) Int. Cl. A61K 45/00; A61P 37/04; G09B 25/00

(52) U.S. Cl. 424/278.1; 434/365

(57) ABSTRACT

A method for increasing natural killer cell activity in an animal by feeding the animal lipoic acid in amounts of from about 10 mg to about 1000 mg per day.

### CROSS-REFERENCE TO RELATED APPLICATIONS

This application is a continuation of International Application No. PCT/US2005/042805 filed Nov. 23, 2005, which claims the benefit of U.S. Provisional Application Ser. No. 60/630,972, filed Nov. 24, 2004, the contents of both of which are incorporated herein by reference.

### BACKGROUND OF THE INVENTION

The present invention generally relates to methods for increasing the immune response in an animal and specifically to increasing natural killer cell activity in an animal.

Due to the constant exposure to an incredible diversity of bacteria, viruses and parasites, most mammals have developed an immune system to defend against such attacks. Natural Killer (NK) cells are important effectors of the innate immune response that have been described on a functional basis as capable of killing tumors or virally infected cells without previous stimulation. Studies have shown that as some mammals age their immune response degrades. The health of their companion animal such as a dog is very important to many people, especially as the animal ages. What is needed is a method to increase NK cell activity in animals, particularly in aging animals.

### SUMMARY OF THE INVENTION

The present invention provides methods for increasing natural killer cell activity in an animal by feeding a natural killer cell activity increasing amount of lipoic acid to the animal, generally in a diet comprising lipoic acid in an amount of at least 50 ppm on a dry weight basis.

In various embodiments, the invention is a new approach for improving the health of aging animals based upon the use of lipoic acid as part of a diet that is fed to the animals. The new approach involves increasing natural killer cell activity in animals, especially dogs, based upon the use of lipoic acid as part of a diet that is fed to the animals.

Further areas of applicability of the present invention will become apparent from the detailed description provided hereinafter. It should be understood that the detailed description and specific examples, while indicating illustrative embodiments of the invention, are not intended to limit the scope of the invention.

### BRIEF DESCRIPTION OF THE DRAWINGS

The present invention will become more fully understood from the detailed description and the accompanying drawings.

FIG. 1 is a graphical representation of the effect of lipoic acid on NK cell activity in old dogs as compared to young and old controls.

The Figure is intended to exemplify the general characteristics of the invention for the purposes of the description of such embodiments herein. This Figure may not precisely reflect the characteristics of any given embodiment and is not necessarily intended to define or limit specific embodiments within the scope of this invention.

### DETAILED DESCRIPTION OF THE INVENTION

The term “animal” means any animal susceptible to or suffering from impaired immune response and in need of increased or improved immune response or an animal that could benefit from an increased immune response. An animal is “susceptible to” a disease or condition if the animal exhibits symptoms that indicate that the animal is likely to develop the condition or disease. An animal is “suffering from” a disease or condition if the animal exhibits symptoms that are indicative that the animal has developed the condition or disease.

The term “older animal” means any animal susceptible to or suffering from impaired immune response and in need of increased or improved immune response or an animal that could benefit from an improved immune response because of age.

The term “single package” means that the components of a kit are physically associated in or with one or more containers and considered a unit for manufacture, distribution, sale, or use. Containers include, but are not limited to, bags, boxes, bottles, shrink wrap packages, stapled or otherwise affixed components, or combinations thereof. A single package may be containers of individual food compositions physically associated such that they are considered a unit for manufacture, distribution, sale, or use.

The term “virtual package” means that the components of a kit are associated by directions on one or more physical or virtual kit components instructing the user how to obtain the other components, e.g., in a bag containing one component and directions instructing the user to go to a website, contact a recorded message, view a visual message, or contact a caregiver or instructor to obtain instructions on how to use the kit.

In one aspect, the present invention provides methods for increasing natural killer cell activity in animals. The methods comprise feeding a natural killer cell activity increasing amount of lipoic acid to the animal. Generally, the lipoic acid is fed to the animal in amounts of greater than 5 mg per day, preferably from about 10 to about 1000 mg per day, most preferably from about 50 to about 500 mg per day. In another aspect, the invention provides compositions for increasing natural killer cell activity in animals. The compositions comprise lipoic acid in amounts of at least 50 ppm, preferably at least 150 ppm. In various embodiments, the methods and compositions are useful for increasing immune function and response in animals, particularly in older animals.

The nutrition and health of companion animals is one of the most important aspects of care, particularly pet care for animals. Many caregivers have a difficult time determining if an animal is receiving a well-balanced and healthy diet. While people are becoming much more aware regarding their own personal nutrition, there is little knowledge of the advanced dietary requirements that an animal must have. Natural Killer (NK) cells are important effectors of the innate immune response that have been described on a functional basis as capable of killing tumors or virally infected cells without previous stimulation. Studies have shown that as some mammals age their immune response degrades. Since the immune response is degrading, an increase in NK cell activity may improve the health of the aging mammal.

The present invention provides a method for feeding an animal, e.g., a companion animal such as a dog, a composition or diet containing lipoic acid to increase or enhance the immune response, particularly when it may be impaired by age, and improve the overall health of the animal. The amount of lipoic acid given to the animal is a non-toxic amount. The lipoic acid may be provided to the animal either as a supplement or contained in a composition, including a diet, fed to the animal. Such a supplement may be in the form of a pill or capsule, a treat or a biscuit, or any other edible form. By “diet”, it is meant the food or drink regularly consumed by the animal. A diet may include supplements consumed by the animal. A diet is considered to have essentially enough nutrients to be life sustaining for the animal. A companion animal diet can be any suitable pet food formula which provides adequate nutrition for the animal. For example, a typical canine diet for use in the present invention may contain from about 8 to 50% fat, about 16 to 50% by weight protein and about 3 to 15% total dietary fiber. In another example, a typical feline diet may contain about 8 to 50% by weight fat and from about 30 to 60% by weight protein. However, no specific ratios or percentages of these or other nutrients are required. A nutrient is any food constituent that helps support life. Nutrients important to an animal's health are known to skilled artisans, e.g., proteins, carbohydrates, fats, fibers, vitamins, and minerals. Water is also vital to an animal's health.

The free radical theory of aging proposes that oxidative stress results in aging and a decrease in the reduced to oxidized ratio of intracellular anti-toxins such as glutathione. Glutathione is prevalent in the liver and is utilized to conjugate xenobiotics for the elimination through the bile duct and eventual elimination via the feces. As such, decreased concentration and activity of glutathione in aged animals may result in the impaired clearance of xenobiotics that contribute to cancer, toxicity and other unwanted effects. In addition, it is known that immune function undergoes senescence with increasing age. Previous studies have shown that GSH:GSSG ratios may be improved in lymphocytes from dogs supplemented with lipoic acid, however, a functional outcome has not been quantified. (See Zicker, S C et al., Veterinary Therapeutics, 3(2): 167-176, 2002). Attempts have been made to improve immune response in companion animals by introducing beta-carotene in combination with other antioxidants. (See U.S. Pat. No. 6,310,090 to Hayek issued Oct. 30, 2001 and U.S. Pat. No. 6,133,323 to Hayek issued Oct. 17, 2000).

R-α-lipoic acid (CAS number 1200-22-2, also known as thioctic acid and 1, 2-dithiolane-3-pentanoic acid) naturally occurs in plant and animal tissues, where it is covalently bound to an ε-amino group of lysine residues. Lipoic acid is commercially available and is produced by companies such as BASF and Cognis. Lipoic acid is commercially available as essentially pure R-α lipoic acid or as racemic mixtures of lipoic acid isomers. In plants, lipoic acid is most abundant in spinach and potatoes while in animal tissues, lipoic acid is most abundant in the kidney and the heart. R-α-lipoic acid was first discovered in 1937 (See Snell et al., Journal Bact. 33; 207, 1937) and was not isolated and characterized until 1951 (See Reed et al. Science 114:94-4, 1951). R-α-lipoic acid maybe synthesized and such methods are well known in the art. (See U.S. Pat. No. 2,890,716 to Reed issued Apr. 18, 1961). R-α-lipoic acid has been classified as an antioxidant and has been used in high dosages as a treatment for Type II diabetes. Studies have shown that mixtures of camitine and lipoic acid may enhance metabolism and alleviate oxidative stress. (See U.S. Pat. No. 5,916,912 to Ames et al. issued Jun. 29, 1999 and U.S. Pat. No. 6,365,622 to Cavayzo issued Apr. 2, 2002). In addition, it has been shown that a companion animal diet comprising lipoic acid among other ingredients appears to inhibit the deterioration of the mental capacity of an aged companion animal. (See U.S. Patent Application Publication Nos. 2002/0076469, 2002/0052402, 2002/0076470, 2000/115710, and 2002/0119182.)

NK cells show antibody-independent cell mediation cytotoxicity against target cells such as virus infected cells and tumor cells. In humans, mice, and rats, it has been suggested that NK cells recognize and bind to a target cell and then release cytotoxic factors to destroy the target cell. Such binding and cytotoxic behavior of NK cells have been reported in canines (see Nariai, Y. et al., J. Bte. Med. Sci. 61 (7): 835-838, 1999), although the mechanism differs from the above animal examples. Studies have shown that mitochondrial oxidation plays a role in the metabolism of lipoic acid. Although the metabolism in humans mainly resembles that observed in mice and rats, the formation of oxidized structures related to tetranorlipoic acid found in canines appears to have no equivalent in humans. In addition, 3-ketolipoic acid, an intermediate in the mitochondrial oxidation of lipoic acid has been reported in plasma samples from rats and humans but has not been found in plasma from canines. (See Schupke, H. et al. Drug Metabolism and Disposition, 29 (6) 855-862, 2001). It appears that the metabolic pathway of α-lipoic acid is different in canines as compared to humans.

Various embodiments of the invention include a method for increasing NK cell activity in an animal, particularly a companion animal. In such embodiments, the method comprises feeding to the animal a composition, e.g., a diet, comprising lipoic acid in an amount of at least 50 ppm on a dry matter basis per day. In other embodiments the method comprises feeding to the animal a diet comprising lipoic acid in an amount of at least 100 ppm on a dry matter basis per day. In still other embodiments, the method comprises feeding to the animal a diet comprising lipoic acid in an amount from about 75 ppm to about 150 ppm on a dry matter basis per day. As used herein, lipoic acid is in a racemic mixture, but other embodiments of the invention may include lipoic acid which is essentially pure R-α-lipoic acid or as a lipoate derivative, mixtures of isomers, salts, esters, amides or combinations thereof (For example see U.S. Pat. No 5,621,177 to Bethge et al. issued Apr. 15, 1997).

In various embodiments of the invention, a companion pet composition or diet comprising at least 50 ppm of lipoic acid, dry matter basis, increases immune response in older dogs. In some embodiments of the invention, the lipoic acid is added to the companion animal's food. In such embodiments, the lipoic acid may be added during the processing of the companion animal food that is then packaged and made available to consumers. Such processes may include extrusion, canning, baking, and the like or any other method or process of producing pet foods that is known in the art. In such processes, the lipoic acid may be contributed by a natural source like an animal or plant component, such as kidney or spinach, or the lipoic acid may be contributed by a synthetically derived source, or the lipoic acid may be contributed by a mixture of natural and synthetic sources. In other embodiments of the invention, lipoic acid may be in a capsule form to be fed to the companion animal. In still other embodiments of the invention, the lipoic acid may be in a powder or in a crystalline which may be added to the animal's food or fed directly to the animal. In various embodiments of the invention, the companion animal diet comprises lipoic acid and other needed nutritional components. In various embodiments of the invention, the companion animal is a dog and in other embodiments of the invention, the companion animal is a cat. Studies have shown that lipoic acid may be ten times more toxic in cats than in dogs. (See Hill, A S et al., J. Anim. Physiol. Anim. Nutr. 88(3-4): 150-156, 2004). In various embodiments of the invention wherein the companion animal is a cat, the diet comprises less than 30 ppm of lipoic acid on a dry weight basis.

In a further aspect, the present invention provides for a use of lipoic acid to prepare a medicament. In another, the invention provides for the use of lipoic acid to prepare a medicament for maintaining and/or improving animal health, e.g., increasing natural killer cell activity in an animal by feeding a natural killer cell activity increasing amount of lipoic acid to the animal. Generally, medicaments are prepared by admixing a compound or composition with excipients, buffers, binders, plasticizers, colorants, diluents, compressing agents, lubricants, flavorants, moistening agents, and other ingredients known to skilled artisans to be useful for producing medicaments and formulating medicaments that are suitable for administration to an animal.

In a further aspect, the present invention provides kits suitable for feeding lipoic acid to an animal. The kits comprise in separate containers in a single package or in separate containers in a virtual package, as appropriate, lipoic acid and at least one of (1) one or more ingredients suitable for consumption by an animal, (2) instructions for how to combine the lipoic acid and other kit components to increase immune response, particularly to produce a composition useful for increasing immune response, and (3) instructions for how to use the lipoic acid and other components of the present invention, particularly for the benefit of the animal. When the kit comprises a virtual package, the kit is limited to instructions in a virtual environment in combination with one or more physical kit components. The kit contains the lipoic acid and other components in amounts sufficient to increase immune response. Typically, the lipoic acid and the other suitable kit components are admixed just prior to consumption by an animal. In one embodiment, the kit contains a packet containing lipoic acid and a container of food for consumption by an animal. The kit may contain additional items such as a device for mixing the lipoic acid and ingredients or a device for containing the admixture, e.g., a food bowl. In another embodiment, the lipoic acid is mixed with additional nutritional supplements such as vitamins and minerals that promote good health in an animal.

In another aspect, the present invention provides a means for communicating information about or instructions for one or more of (1) using lipoic acid to increase immune response, (2) admixing lipoic acid with the other components of the present invention, (3) feeding lipoic acid to an animal, alone or in combination with the other elements of the present invention, and (4) using the kits of the present invention for increasing immune response comprising a document, digital storage media, optical storage media, audio presentation, or visual display containing the information or instructions. In certain embodiments, the communicating means comprises a document, digital storage media, optical storage media, audio presentation, or visual display containing the information or instructions. Preferably, the communication means is a displayed web site or a brochure, product label, package insert, advertisement, or visual display containing such information or instructions. Useful information includes one or more of (1) methods and techniques for combining and feeding the lipoic acid and/or other components and (2) contact information for animals or their caregivers to use if they have a question about the invention and its use. Useful instructions include amounts for mixing and administration amounts and frequency. The communication means is useful for instructing on the benefits of using the present invention and communicating the approved methods for feeding the invention to an animal.

This invention is not limited to the particular methodology, protocols, and reagents described herein because they may vary. Further, the terminology used herein is for the purpose of describing particular embodiments only and is not intended to limit the scope of the present invention. As used herein and in the appended claims, the singular forms “a,” “an,” and “the” include plural reference unless the context clearly dictates otherwise. The terms “comprise”, “comprises”, and “comprising” are to be interpreted inclusively rather than exclusively.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

All patents, patent applications, and publications mentioned herein are incorporated herein by reference to the extent allowed by law for the purpose of describing and disclosing the compositions, compounds, methods, and similar information reported therein that might be used with the present invention. However, nothing herein is to be construed as an admission that the invention is not entitled to antedate such disclosure by virtue of prior invention.

### EXAMPLES

This invention can be further illustrated by the following examples of preferred embodiments thereof, although it will be understood that these examples are included merely for purposes of illustration and are not intended to limit the scope of the invention unless otherwise specifically indicated.

### Example 1

The study involves three groups of dogs: Group 1—young dogs on a controlled food, Group 2—old dogs on a controlled food, and Group 3—old dogs on a dry food fortified with 150 ppm of lipoic acid on a dry matter basis. The dogs are beagles and Group 1 consists of 10 beagles with the average age of 5.1 years old, Group 2 consists of 10 beagles with an average age of 11.8 years old, and Group 3 consists of 10 beagles with an average age of 11.3 years old. The dogs from all three groups are fed the controlled food for a two week period prior to intervention. After a two week period, Group 3 is transferred to a diet of dry food fortified with 150 ppm of lipoic acid on a dry matter basis. During the two week period on the control, samples are taken from all dogs and chemical and biological analysis is completed on the samples to establish base line data. Then three groups of dogs eat their respective diets for a six week period of time and after this time period, samples are taken from all of the dogs in the study and chemical and biological analysis is completed on the samples. Baseline data is compared to the data taken at the end of the six week study.

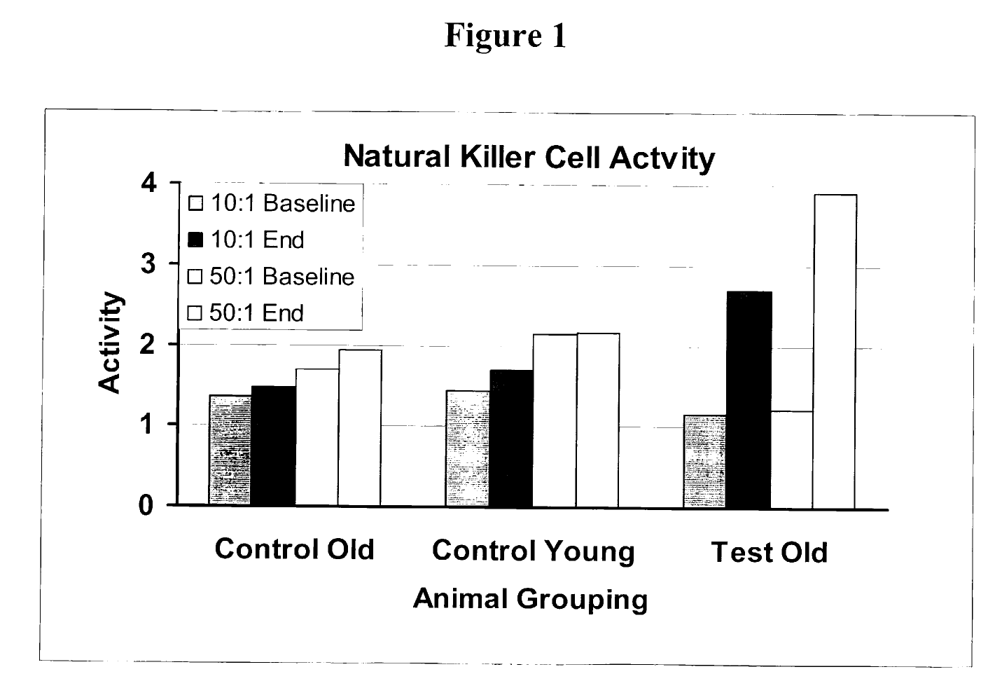

Results as in FIG. 1 show the dogs in Group 3 which included a diet with 150 ppm of lipoic acid on a dry matter basis have increased NK cell activity.

In the specification, there have been disclosed typical preferred embodiments of the invention and, although specific terms are employed, they are used in a generic and descriptive sense only and not for purposes of limitation, the scope of the invention being set forth in the following claims. Obviously many modifications and variations of the present invention are possible in light of the above teachings. It is therefore to be understood that within the scope of the appended claims the invention may be practiced otherwise than as specifically described.

What is claimed is:

1. A method for increasing natural killer cell activity in an animal comprising feeding a natural killer cell activity increasing amount of lipoic acid to the animal.

2. The method of claim 1 comprising feeding the lipoic acid in an amount of greater than 50 mg per day.

3. The method of claim 1 comprising feeding the lipoic acid in an amount of from about 10 to about 1000 mg per day.

4. The method of claim 1 wherein the animal is a companion animal.

5. The method of claim 1 wherein the companion animal is a canine.

6. The method of claim 1 wherein the lipoic acid is in capsule form.

7. The method of claim 1 wherein the lipoic acid is in powder form.

8. The method of claim 1 wherein the lipoic acid is in crystalline form.

9. The method of claim 1 wherein the lipoic acid is part of the animal's daily diet.

10. The method of claim 9 wherein the daily diet comprises lipoic acid in an amount of greater than 50 ppm on a dry matter basis.

11. The method of claim 1 wherein the lipoic acid is fed to the animal in a composition suitable for consumption by the animal.

12. The method of claim 1 wherein the animal is an older animal.

13. A kit suitable for feeding lipoic acid to an animal comprising in separate containers in a single package or in separate containers in a virtual package, as appropriate at least one lipoic acid and at least one of (1) one or more ingredients suitable for consumption by an animal, (2) instructions for how to combine the lipoic acid and other kit components to increase immune response or natural killer cell activity, and (3) instructions for how to use the lipoic acid and other components of the present invention.

14. A means for communicating information about, or instructions for, one or more of (1) using lipoic acid to increase immune response or natural killer cell activity, (2) admixing lipoic acid with the other components of the present invention, (3) feeding lipoic acid to an animal, alone or in combination with the other elements of the present invention, and (4) using the kits of the present invention for increasing immune response or natural killer cell activity, the means comprising a document, digital storage media, optical storage media, audio presentation, or visual display containing the information or instructions.

15. The means of claim 14 selected from the group consisting of a displayed web site, brochure, product label, package insert, advertisement, or visual display.

16. A composition suitable for increasing immune response or natural killer cell activity in an animal comprising: a life sustaining amount of nutrients; and greater than 50 ppm of lipoic acid.

17. The composition of claim 16 wherein the composition is a food composition.

18. The composition of claim 17 wherein the food composition is suitable for a companion animal.

19. The composition of claim 17 wherein the food composition is suitable for a canine.

20. The composition of claim 17 wherein the food composition is extruded or canned.

21. A use of a composition that comprises lipoic acid to prepare a medicament for increasing the immune response by increasing natural killer cell activity in an animal.

22. A use of a composition that comprises lipoic acid to prepare a medicament for increasing natural killer cell activity in an animal.

23. A method for increasing the immune response in an animal comprising feeding an immune response increasing amount of lipoic acid to the animal.
